(19) United States
(12) Patent Application Publication
(10) Pub. No.: US 2023/0226121 A1
(43) Pub. Date: Jul. 20, 2023

(54) Honey Foam

(71) Applicant: Mark Edward Fenzl, Sarasota, FL (US)

(72) Inventor: Mark Edward Fenzl, Sarasota, FL (US)

(21) Appl. No.: 17/725,587

(22) Filed: Apr. 21, 2022

Related U.S. Application Data
(60) Provisional application No. 63/361,740, filed on Jan. 15, 2022.

(51) Int. Cl. A61K 35/644; A61K 9/00; A61K 9/12; A61K 36/18; A61K 36/899; A61K 31/167; A61K 36/79; A61K 36/61; A61K 33/40; A61K 33/00

(52) U.S. Cl. CPC A61K 35/644; A61K 9/0014; A61K 9/122; A61K 36/18; A61K 36/899; A61K 31/167; A61K 36/79; A61K 36/61; A61K 33/40; A61K 33/00

(57) ABSTRACT

The present invention relates to an improved wound product that is equivocal to manuka honey in antimicrobial and wound healing effectiveness, but may be mixed with a therapeutic anesthetic, may have a fragrance, may be stored in a spray dispenser, may be used with a straw, and is applied as a foam. This provides the option of a straw being attached to the dispenser hole for dispensing honey foam for deep tunneling wound. It may also be sprayed directly on the wound or burn, or be sprayed on a sterile sponge for application.

### CROSS-REFERENCE TO RELATED APPLICATIONS

This application claims the benefit of U.S. Provisional Application 63/360,034, filed on Aug. 28, 2021. The above application is incorporated herein in reference to combining honey with hydrogen peroxide or carbon dioxide.

This application claims the benefit of U.S. Provisional Application 63/361,740, filed on Jan. 15, 2022. The above application is incorporated herein in reference to mixing an anesthetic with wound honey.

### FIELD

The present disclosure relates to a medical product and, more particularly, a honey foam. This honey foam may be mixed with anesthetics in a spray applicator with or without a straw for contaminated wounds, non-contaminated wounds, decubitus ulcers, deep ulcers, burns, and pre-surgical scrub.

### BACKGROUND

This section provides background information related to the present disclosure which is not necessarily prior art.

The present invention relates to an improved wound product that is equivocal to manuka honey in antimicrobial and wound healing effectiveness, but may be mixed with a therapeutic anesthetic, may be stored in a spray dispenser, may be used with a straw, and is applied as a foam. Manuka Honey comes from Australia and New Zealand because that is where Leptospermum scoparium trees grow. Manuka honey has been studied for its antimicrobial and wound healing properties and honey containing an appropriate amount of methylglyoxal has been approved to be used as a wound honey. The problem with Manuka Honey is that it is limited in location to these areas of the world, there is a limit to the production of Manuka Honey, and it is very expensive. Because of cost, many people can not afford Manuka Honey. Honey has been used for wound healing and as a natural skin moisturizer. It has been known for decades that moist wounds heal faster than dry wounds. Table honey contains hydrogen peroxide instead of methylglyoxal. It has been discovered by this inventor that adding hydrogen peroxide to honey creates a semi-solid foam. Hydrogen peroxide adds antimicrobial effect to the honey and foam requires a smaller quantity of honey to cover a larger surface area, allowing for more coverage with less honey. The foam honey may also be used for deeper wounds, such as tunneling decubitus ulcers. Wound honey not only protects wounds from infections, but maintains an ideal moisture content. The honey may be mixed and stored with other natural antimicrobial sweeteners, such as molasses, syrup, artificial honey, or nectar. It may also be mixed with natural or synthetic anesthetic or fragrance.

Two ways a product is protected from microbial spoilage are water activity level and acidity. Products with low water activity level do not have enough water for bacteria or fungus reproduce and cause damage. Acidity or low pH damage microbes, preventing them from damaging the product. Honey naturally has a low water activity level and acidity. By adding ingredients with low water activity and acidity, such as a non-limiting example of oxalic acid, further protects the product.

Honey is naturally preserved with hydrogen peroxide, polyphenols, and organic acids. Although this combination will prevent bacteria from growing in honey, it may not necessarily be bactericidal. Some honeys with high levels of hydrogen peroxide, such as honeydew honey, are bactericidal at lower concentrations than manuka honey. There is a synergistic bactericidal effect between organic acids and hydrogen peroxide. Honey contains a wide variety of organic acids. Supplemental hydrogen peroxide may be added to honey for a bactericidal effect. Please see Table 1.

TABLE 1
Synergistic bactericidal combination of hydrogen peroxide
and organic acids in percentage.

| | | | | MBC combination |
|---|---|---|---|---|
| | | | MBC | Acid / H2O2 |
| | | MBC | hydrogen | In Bovine albumin + |
| Acid | Bacterial Strain | acid | peroxide | yeast extract 1% |
| Formic | Enterococcus hirae | 5% | 12.5% | 0.625% / 3.12% |
| Formic | Staphylococcus | 1.25% | 3.12% | 0.312% / 1.56% |
| | aureus | | | |
| Formic | Listeria | 1.56% | 6.25% | 0.625% / 0.78% |
| | monocytogenes | | | |
| Formic | Pseudomonas | 0.025% | 1.56% | 0.025% / 0.39% |
| | aeruginosa | | | |
| Acetic | Pseudomonas | 0.625% | 1.56% | 0.625% / 0.39% |
| | aeruginosa | | | |

Anesthetics, such as lidocaine also has an antimicrobial effect.

TABLE 2
Minimal Inhibitory Concentration of lidocaine

| | MIC |
|---|---|
| Microorganism | Mg/ml |
| Staphylococcus aureus ATCC 29213 | 20 |
| Escherichia Coli ATCC 25922 | 5 |

Some phytochemicals also have an anesthetic effect.

TABLE 3
Phytochemicals with local anesthetic activity.

| Class | Phytochemical |
|---|---|
| Ester | Cocaine |
| Acyclic Monoterpinoid | Linalool |
| Acyclic Monoterpinoid | Linalyl acetate |
| Acyclic Monoterpinoid | Geranial |
| Acyclic Monoterpinoid | Neral |
| Acyclic Monoterpinoid | Citronellol |
| Acyclic Monoterpinoid | Citronellal |
| Acyclic Monoterpinoid | Geraniol |
| Acyclic Monoterpinoid | Geranyl acetate |
| Monocyclic Monoterpinoid | Menthol |
| Monocyclic Monoterpinoid | Cymene |
| Monocyclic Monoterpinoid | Thymol |
| Monocyclic Monoterpinoid | Carvacrol |
| Monocyclic Monoterpinoid | Terpineol |
| Monocyclic Monoterpinoid | Terpinene |
| Monocyclic Monoterpinoid | Menthone |
| Monocyclic Monoterpinoid | Carvone |
| Monocyclic Monoterpinoid | Pulegone |
| Monocyclic Monoterpinoid | Limonene |
| Monocyclic Monoterpinoid | Eugenol |
| Bicyclic Monoterpinoid | Eucalyptol |
| Bicyclic Monoterpinoid | Cineol |
| Bicyclic Monoterpinoid | Pinene |
| Bicyclic Monoterpinoid | Fenchone |
| Bicyclic Monoterpinoid | Borneol |
| Bicyclic Monoterpinoid | Bornyl acetate |
| Bicyclic Monoterpinoid | Camphene |
| Bicyclic Monoterpinoid | Cinnamaldehyde |
| Bicyclic Monoterpinoid | Anethole |
| Bicyclic Monoterpinoid | Estragole |
| Sesquiterpenoid | Furanodiene |
| Sesquiterpenoid | Methoxyfuranoguaia |
| Sesquiterpenoid | Caryophyllene |
| Sesquiterpenoid | Nerolidol |
| Flavonol | Quercetin |
| Flavanol | Catechin |
| Flavanol | Epicatechin |
| Flavanol | Epigallocatechin |
| Flavanol | Epigallocatechin gallate |
| Anthocyanidin | Pelargonidin |
| Anthocyanidin | Cyanidin |
| Anthocyanidin | Delphinidin |
| Isoflavone | Genistein |
| Isoflavone | Daidzein |
| Stilbenoid | Reservatrol |

Advantageously, non-limiting examples of use of this product may include a spray bottle with a dispensing straw for deep wounds. The product may comprise hydrogen peroxide at a concentration between 0.01% and 10%, anesthetic at a concentration between 0.1% and 10%, and fragrance at a percentage between 0.01% and 3%. Other embodiments may include wound honey in a whipped topping dispenser, a honey foam dispenser with a sponge top, or a sponge containing honey foam for bums, exposed wounds, contaminated wounds, tunnelling wounds, or surgical scrub.

### SUMMARY OF THE INVENTION

In concordance with the instant disclosure, this invention is an improved natural honey foam or honey substitute foam product that may be used as a medical product. An anesthetic and/or fragrance may be added as needed and the product may be dispensed from a straw tipped applicator, sponge tip applicator, or sponge for wounds, burns, deep wounds, or pre-surgical scrub. This product may be equivalent in antimicrobial and wound healing efficacy to Manuka honey, but may cost less. One example of several potential medical products that does not limit this patent application, is sterile medical honey admixed with 0.1%-4% lidocaine, 0.01-0.05% clove essential oil, and 0.01-1% star anise essential oil, dispensed from a pressurized spray bottle containing 0.01%-1% hydrogen peroxide. A variation is a sterile medical honey and 6.5% pomegranate syrup product in a whipped dispenser with nitrous oxide. The product may be dispensed directly into a decubitus ulcer by pushing the dispenser nozzle to one side. Another embodiment is a wound honey in a foam-top dispenser. When the handle is squeezed, hydrogen peroxide is released into the honey and foam honey comes out of the dispenser. A fourth example may be a sterile honey pre-surgical scrub packaged in a sealed sponge with a capsule containing hydrogen peroxide. When ready for use, the sponge is unwrapped from the sterile wrapper, the hydrogen peroxide in the capsule is squeezed releasing the hydrogen peroxide into the sponge, and the honey foam is applied to wounds, burns, or as a pre-surgical scrub.

These products may contain an artificial honey, molasses, syrup, or nectar for better antimicrobial effect or lower cost. They may contain a synthetic or natural anesthetic to decrease pain. They may contain a synthetic or natural fragrance that can create a pleasant scent and hide odor. There is a significant decrease in cost due to use of a less expensive honey. A multipurpose wound honey foam/burn honey foam/surgical sanitizer honey foam will save time and money in the hospital, burn center, surgical center, or wound center because it may be used in multiple departments.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

The present disclosure relates to a hydrogen peroxide that is mixed in with a honey or honey substitute, as described in greater detail herein below.

One example of several potential medical products that does not limit this patent application, is sterile medical honey admixed with 4% lidocaine, 0.03% clove essential oil, 0.02% star anise essential oil, and 1% hydrogen peroxide dispensed from a spray dispenser. The dispenser, as one of multiple potential examples, is a 200 ml pressurized spray bottle with a straw. This provides the options of a straw being attached to the dispenser hole for dispensing honey foam for deep tunneling wounds. The straw may be removed to spray directly on the wound or burn, or sterile sponge for application.

A variation, that does not limit this patent, is a sterile honey 93.5% and sterile pomegranate syrup 6.5% product in a 6.5 ounce whipped dispenser with nitrous oxide. The product may be dispensed directly into a decubitus ulcer by pushing the dispenser nozzle to one side.

Another embodiment, that does not limit this patent, is a wound honey in a 3.5 ounce sponge-top dispenser. When the handle of the flexible, plastic dispenser is squeezed, hydrogen peroxide is released from the bottom of the dispenser into 100% Leptospermum honey and honey foam comes out of the dispenser to saturate the sponge for application.

A fourth example, that does not limit this patent, is a sterile buckwheat honey soaked in a 2½″ X 3½″ pre-surgical scrub sponge containing a capsule with 50 ml hydrogen peroxide embedded in the sponge. When ready for use, the sponge is unwrapped from the sterile wrapper, the hydrogen peroxide in the capsule is squeezed releasing the hydrogen peroxide into the sponge, and the honey foam is applied to wounds, burns, or as a pre-surgical scrub.

It is desirable for the supplement to have a low water activity level. Honey naturally has a low water activity level containing approximately 17% water. Other additional humectants may include nitric acid, dextrose, fructose, glycerol, glycine, glucose, malic acid, salt, sorbitol, sucrose, and tartaric acid. A skilled artisan may select any suitable humectant, as desired. For example, the supplement can have a water activity level of 0.86.

It is also desirable for a product to have a low pH as this inhibits microbes. Honey typically has a pH between 3.5 and 5.5. Acids lower the pH. Some acids, such as formic acid, acetic acid, oxalic acid, nitric acid, lactic acid, succinic acid, tartaric acid, citric acid, propionic acid, sulfhuric acid, benzoic acid, sorbic acid, sulphamic acid, phosphoric acid, mandelic acid, and adipic acid have synergistic antimicrobial effects with hydrogen peroxide against bacteria. Hydrogen peroxide is naturally found in non-mauka type honeys.

Additionally, as may be presented in the claims herein, the language “consisting of” is intended to exclude any ingredient not specified in the claim. Accordingly, the supplement consisting of certain ingredients described hereinabove includes only those ingredients.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

1. A wound, burn, or pre-surgical scrub product comprising at least one of honey, artificial honey, molasses, nectar, and syrup that is admixed with hydrogen peroxide or carbon dioxide at a concentration sufficient to cause the product to form a foam.

2. A product in claim #1 that is stored in a pressurized container.

3. A product in claim #1 that may be applied using a straw.

4. A product in claim #1 that is admixed with an anesthetic.

5. A product in claim #1 that is admixed with an acid.

6. A product in claim #1 that has a water activity level less than 0.87.

7. A honey foam combined with a natural or synthetic anesthetic.

8. A product in claim #7 that is admixed with lidocaine at a concentration less than 5.1%.

9. A product in claim #7 that is admixed with clove essential oil at a concentration less than 0.06%.

10. A product in claim #7 that is admixed with star anise essential oil at a concentration less than 0.5%.

11. A product in claim #7 that is admixed with 4% lidocaine and 0.03% clove essential oil.

12. The process of mixing hydrogen peroxide, carbon dioxide, or nitrous oxide with at least one of honey, artificial honey, molasses, nectar, and syrup to create a foam product.

13. The process in claim #12 to create honey foam.

14. A product created in claim #12 from ingredients stored in a sponge or sponge-top dispenser.
